(19) United States
(12) Patent Application Publication
(10) Pub. No.: US 2022/0005590 A1
(43) Pub. Date: Jan. 6, 2022

(54) SYSTEM FOR A CUSTOMER AND MEDICAL SYSTEM INTERFACE

(71) Applicants: Trace Lee Polanco, Chula Vista, CA (US); Sergio Oliveros, Chula Vista, CA (US)

(72) Inventors: Trace Lee Polanco, Chula Vista, CA (US); Sergio Oliveros, Chula Vista, CA (US)

(21) Appl. No.: 15/674,533

(22) Filed: Aug. 11, 2017

(51) Int. Cl. G16H 40/20; G16H 10/60; G06Q 10/10; G06F 16/27

(52) U.S. Cl. CPC G16H 40/20; G16H 10/60; G06Q 40/08; G06F 16/27; G06Q 10/1095

(57) ABSTRACT

A new and improved system and method for a customer and medical system interface is described herein.

### CROSS-REFERENCE

The present application claims benefit of the Provisional Application Ser. No. 62/483,360, entitled “A New and Improved Method for a Customer and Medical System Interface” filed on Apr. 10, 2017, which is herein incorporated by reference in its entirety.

### BRIEF DESCRIPTION OF THE FIGURES

For a more complete understanding of the present invention, including its features and advantages, reference is now made to the detailed description of the invention taken in conjunction with the accompanying drawing in which:

FIG. 1 is an architectural overview of the first embodiment;

FIG. 2 illustrates several models that are included in the first embodiment;

FIG. 3 illustrates several other models that are included in the first embodiment;

FIG. 4 also illustrates several other models that are included in the first embodiment;

FIGS. 5-6 are flow diagrams of one aspect of the embodiment; and

FIGS. 7-8 are flow diagrams of another aspect of the embodiment.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

First, an architectural overview of an embodiment of the invention is described herein referred to as Enhanced Access Platform. This embodiment uses a number of different architectural views to depict different aspects of the system. It includes several architectural decisions employed by the system. The concepts of the embodiment depicted throughout apply to multiple industries. Any references to a medical services portal are intended as examples, not as the unique manner in which the system can be utilized.

The following paragraphs provide a description of components as it pertains to this embodiment, a system that provides a business with the ability to schedule, monitor, maintain and charge customers for services rendered in person or virtually.

This embodiment includes a Portal, also referred throughout as the EAP Portal. In order to provide complete documentation of the aspects of the architecture of the EAP Portal, the following paragraphs are divided into sections that adhere to the “4+1 architectural view model” whereby the architecture is described based on multiple, concurrent views meant to cover the end-users, developers, and project managers. The 4 typical views of the model are: 1) Development—represents the software developer's point of view; 2) Logical—focuses heavily on the functionality of the system; 3) Physical—describes the system from a system's engineer perspective, focusing on physical and external connection; and 4) Process—focuses on the abilities of the software and its growth capacity.

This embodiment described herein includes views such as: use-case, logical, process, deployment, implementation and data. Examples are shown in UML diagrams to improved and readability and maintain the flow of the description of the embodiment.

While the current embodiment described herein runs on Apache via the Phusion Passenger module, using Ruby on Rails, other embodiments of the invention can run on other platforms as well. In addition, this version of the embodiment complies with HIPAA regulations in order to protect PHI (Personal Health Information) for those customers who want to use the portal in the health industry.

Moreover, this embodiment adheres to the HITECH act to comply with security standards for those customers who want to use the portal in the health industry. Further, this embodiment ensures all credit card or other financial transactions are transmitted in a secure manner to comply with PCI. In addition, this embodiment includes use authentication via a login with username and password. Specifically, this embodiment also includes use authorization of every resource by their specific role. Other aspects of this embodiment include: 1) Administrators have access to all resources and can define roles and users as needed, giving permissions by function to every user of the system; 2) Confidentiality is ensured by providing encryption via SSL on every area of the system; 3) Every sensitive action is recorded in order to provide accurate auditing; and 4) Many credit theft actions are eliminated by not storing credit card information in a local database.

Moreover, this embodiment of the EAP Portal handles persistence by using a MySQL relational database and ruby on rails Model/View/Controller Active Record relational mapping.

Further, this embodiment of the EAP Portal is offered to clients as a cloud based solution. Therefore, high availability is required to allow our customers' clients to access the application at various times and days. The Apache/Phusion Passenger/Ruby on Rails solution allows us to target system availability at an average of 23 hours a day 7 days a week.

Additionally, this embodiment of the EAP Portal can be deployable in 60 minutes or less to another server in the event the current server encounters software, operating system or hardware problems. The newly deployed server has the capacity to port the latest known data.

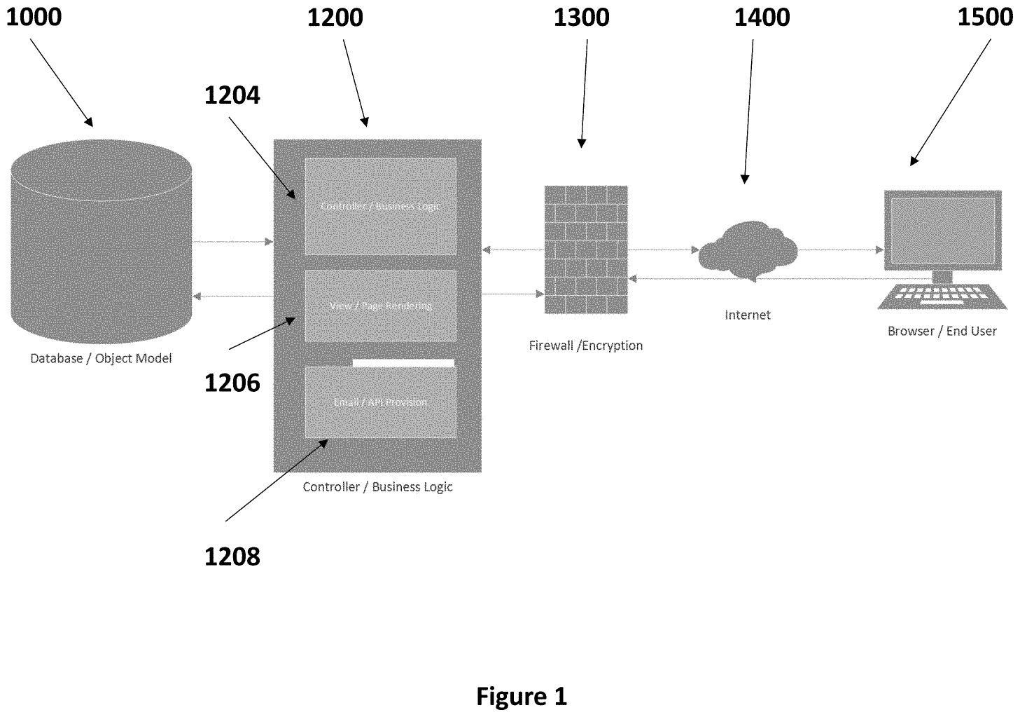

Now referring to the embodiment depicted in FIG. 1, a Database (aka Object Model) 1000 stores information including users, roles, services, appointments, events, schedules, organizations, categories, products, product types, sessions, health care credits, user transaction histories, user credit usage histories, locations, web conferences, web conference rooms and web conference signals. Although the content of each Object Model 1000 is described in latter paragraphs. However, the Object Model 1000 exchanges information with the Controller (aka business logic model) 1200 which deals with interpretation of the data information, giving the data context and produces results for the appropriate recipient. In addition, the Controller 1200 includes a controller business logic submodule 1206, a view/page rendering submodule 1206, and an email/API provisioning submodule 1208. Moreover, in this embodiment, the Controller 1200 utilizes core functionality of the system and responds to requests from the end user 1500 via the Internet 1400 and an appropriate firewall 1300.

Further, the Controller 1200 includes application controllers. The application controllers in this embodiment include: 1) Add-on Services: Services that can be offered while a main service is taking place; 2) Affiliate Services: Services offered by affiliate partners; 3) Application: Logic for specifying application-wide behaviors; 4) Appointment Steps: Logic for specifying the sequence of events of an appointment; 5) Appointments: Logic for Creating, Reading, Updating and Deleting appointments; 6) Calendar Events: Logic for dealing with schedules by office staff; 7) Calendars: Logic for Creating, Reading, Updating and Deleting calendars; 8) Expert Block Dates (Aliased Doctor Blocked Dates); 9) Home (For the home page); 10) Mailboxes: Logic for receiving and storing private messages; 11) Organization Categories: Logic for Creating, Reading, Updating and Deleting organization categories; 12) Organizations: Logic for Creating, Reading, Updating and Deleting organizations; 13) Passwords: Logic for Creating, Reading, Updating and Deleting user passwords; 14) Private Messages: Logic for Creating, Reading, Updating and Deleting private messages; 15) Product Types: Logic for Creating, Reading, Updating and Deleting product types; 16) Registrations: Logic for dealing with user new account management; 17) Policies: Logic for Creating, Reading, Updating and Deleting company policies; 18) Services: Logic for Creating, Reading, Updating and Deleting main services offered; 19) Users: Logic for dealing with user actions and their particular roles; 20) Visit Locations: Logic for Creating, Reading, Updating and Deleting places where services are offered; 21) Visit Types: Logic for Creating, Reading, Updating and Deleting the types of visits provided by the company; 22) Web Conference: Logic for Creating, Reading, Updating and Deleting web conferences; and 23) Web Conference Rooms: Logic for Creating, Reading, Updating and Deleting virtual rooms.

In this embodiment, the design component for rendering the end user views resides in the server. However, it is important to note that the way in which the site is displayed depends largely on the user's browser and internet connection. In this embodiment, the following end user configurations are contemplated: 1) up-to-date popular browser. Chrome, Firefox, Edge, Safari, Opera are the platforms that the system were tested; 2) Internet connection; and 3) session Cookies available for processing on the client's browser.

In this embodiment, several models are included within the system that help organize all the data contained and exchanged between applications and/or modules. Each of these models include individual records. Further, each record contains fields. In this embodiment, each record contains the following fields: 1) Created at: Indicates the date the record was initially entered in the database; and 2) Updated at: Indicates the date the record was last modified.

Furthermore, several records contain 1) deleted; and 2) deleted_at fields. One purpose for these fields is to ensure data remains beyond deletion, thus providing enhanced auditing.

Additionally, in this embodiment, models for describing services can be added while a main service is taking place. Some examples include: 1) Adding a vaccine while an initial visit is in place; and 2) Adding a pet's nail clip while a grooming visit is in place.

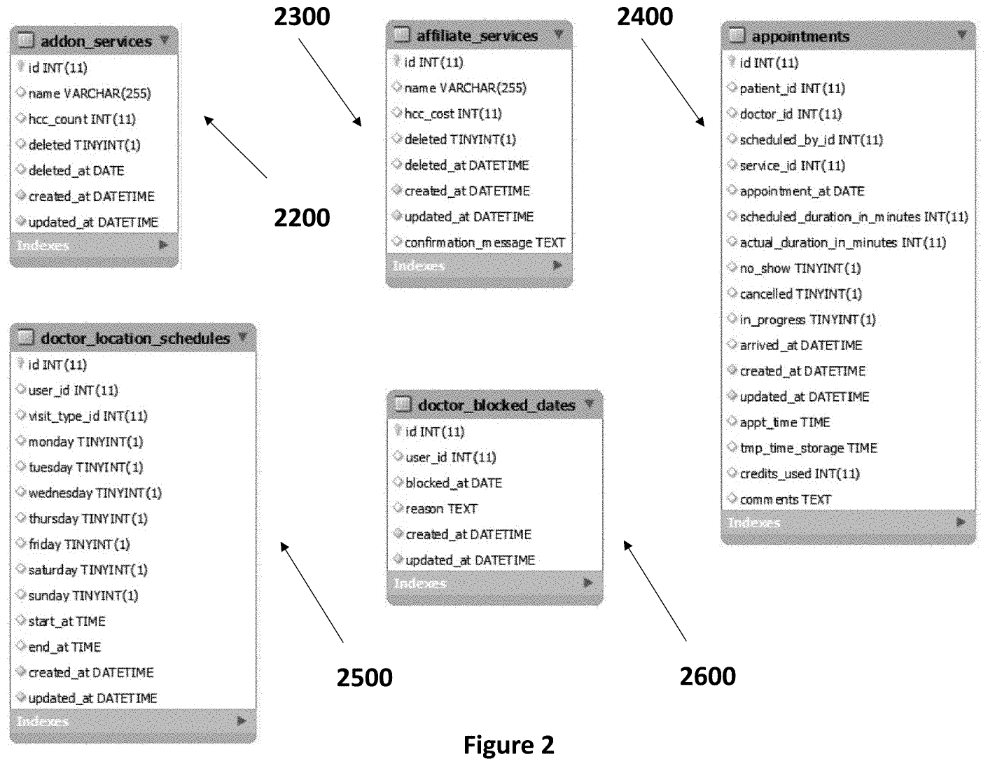

One example model 2200 for an added-on service is depicted in FIG. 2.

Moreover, a model for affiliate services 2300 can be used for describing services that can be offered by affiliate partners. Examples include: 1) Imaging services to a medical office; and 2) Massage/Acupuncture to a Spa.

An additional Appointments Model 2400 is also depicted and used for describing a visit to an expert (doctor) by a client (patient) at a given time. The appointment is one of the central pieces of the architecture, as it defines the type of visit to be conducted, the duration of that visit, and the status of that visit. Of high importance is the number of credits used for the visit. Some example appointments include: 1) visits to a medical office to see a doctor; 2) visits to a hair salon; and 3) visits to a veterinary.

Another model 2600 is used for specifying the dates that an expert is not available to offer services. This model 2600 allows experts to have the flexibility to determine their schedule on dates that would be otherwise working days. A couple of examples are: 1) blocking an entire day due to a conference by a doctor; and 2) blocking an entire day due to a personal matter.

Another model 2600 can be utilized for specifying available dates and locations for an expert. This model 2600 provides the flexibility to customize each expert/doctor's services and locations based on preference. Some examples are: 1) On site visit Monday thru Friday 8 AM to 5 PM for a given doctor; 2) Home visit Wednesdays Noon to 3 PM by a given doctor; and 3) Offsite free dental cleaning visits on Mondays 6 AM to 10 AM.

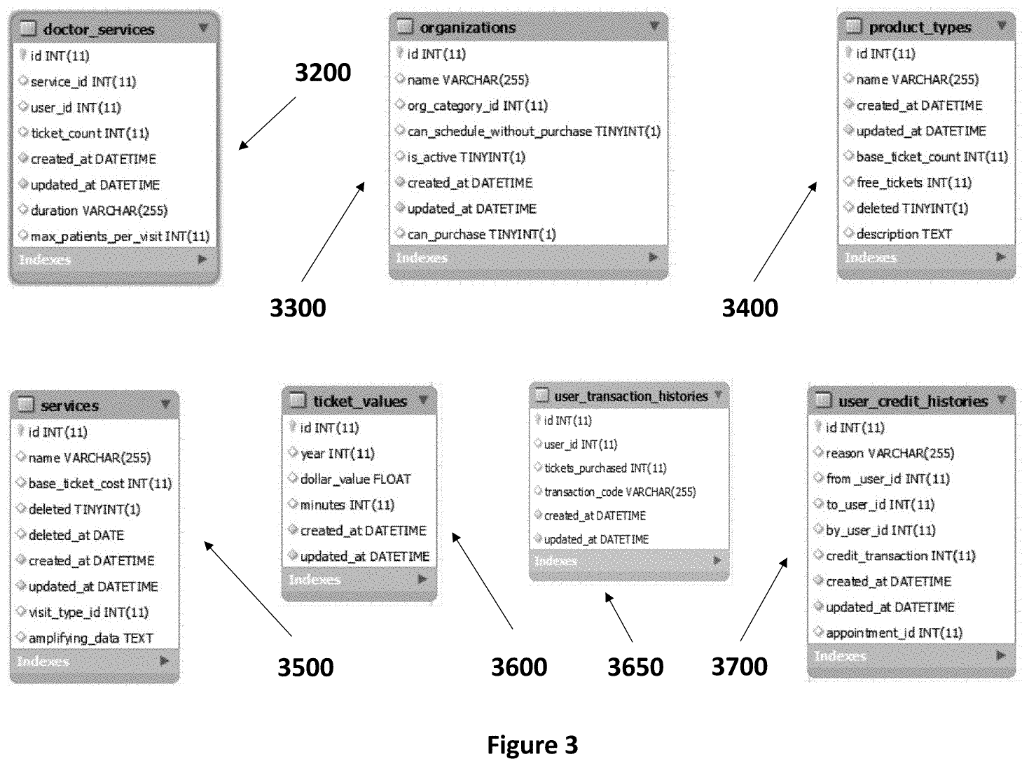

FIG. 3 includes a model 3200 for describing the services that a given expert is willing to provide while using the EAP Portal. This model 3200 includes the number of credits the expert wants to charge and the number of people that can be seen at the same time for that given service. Some other examples are: 1) Home Visits by a given doctor; 2) Parasite removal by a given veterinarian; and 3) Color highlights by a given hair dresser.

Another model 3300 is used for describing the main entity to which a particular client belongs. This model 3300 provides the definition of purchasing with or without credits and the active state of that organization within the business using the EAP Portal. The model is also of value to note that organization assignments are tracked via the organization assignment histories, which helps keep the ID of the user and the ID of the organization along with the dates of the assignment. Some examples are: 1) Insurance Company (Aetna, Blue Shield, etc); 2) VIP; and 3) Independent Entity (SCMG, AAA, SBPMG, OS5, etc.).

Another model 3400 is used for defining the different business packages to offer clients. Some examples are: 1) Platinum Package (Buy 10 credits, get 5 credits free); and 2) Ultimate Spa Package (Buy 100 credits, get 10 credits free).

Another model 3500 is used for describing the main services that are offered by the business using the EAP Portal. Some examples are: 2) Initial Visit to a medical office; 2) Follow up visit to your doctor; 3) Grooming service for a pet store; and 4) Root canal service for a dental office.

Another model 3600 is used for describing the monetary cost of a credit, based on a given year. An example is $50 per credit in 2017

Yet another model 3650 is used for describing the utilization of credits based on who initiated it, who was the beneficiary and who was the grantor. Some examples are: 1) Purchase of credits online; 2) Purchase of credits on-site; and 3) Transfer of credits by office staff from client 1 to client 2.

Another model 3700 is used for describing the purchase of credits, and the date it takes place. Note that is this embodiment, none of the credit card information is stored locally. However, an example of what type of information is kept is if the user made a purchase of 50 credits on Mar. 1, 2017.

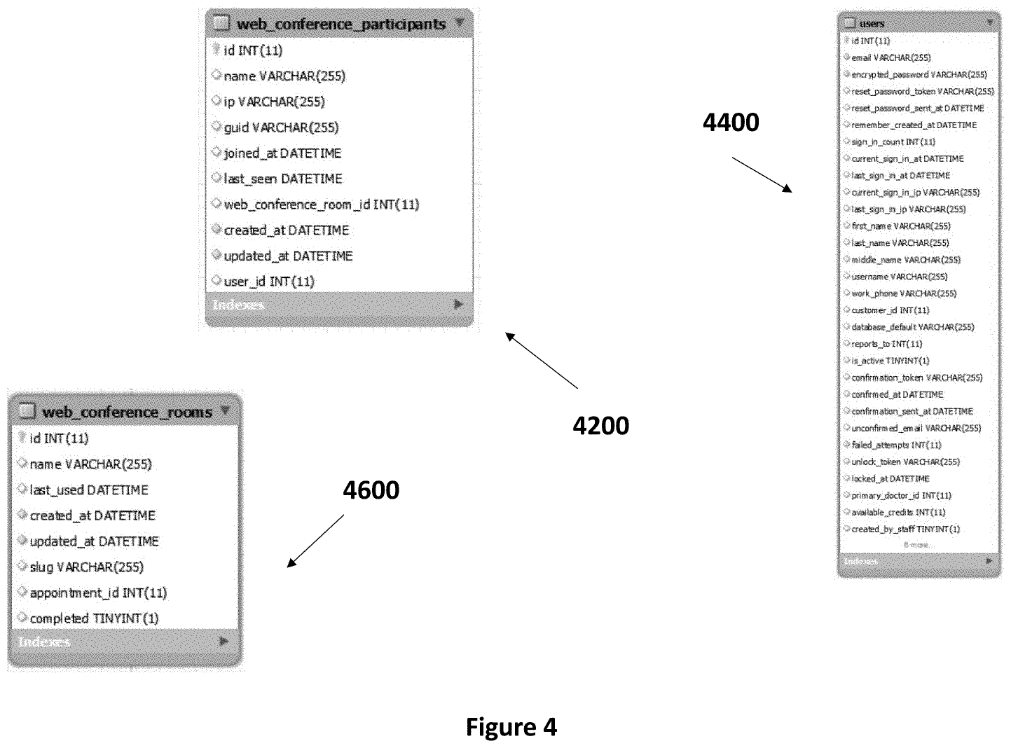

Additional models are illustrated in FIG. 4. However, one model, although not depicted, can be used for describing the physical place where the visit takes place. Some examples are: 1) an address (769 Medical Center Ct); and 2) a place of interest (San Diego Country Club, World Gym).

Another model 4200 is used for describing the individuals participating in a web visit. This model 4200 can be used to provide the proper additional security to web consults (Telehealth). Some examples are: 1) Web Consult between Dr. Smith and Patient Schmitt; and 2) Web visit between school counselor Smith and Student Schmitt.

Another model 4600 is used for describing a virtual (web) room. A virtual room is defined as the space for a visit between 2 individuals who have agreed to meet via an appointment. One example includes web Conference Room March 12 Smith-Schmitt.

One other model 4400 depicted is main component of this embodiment. The user model 4400 defines persons that have an account in the EAP Portal. Through other interfaces, the user model 4400 has roles, access, appointments, schedules, dates to block and other attributes.

The specific type of business that the EAP Portal can handle varies greatly, but the process for providing the service remains the same. The diagrams below have a doctor/patient flow, but the users can be replaced to be any expert with any client.

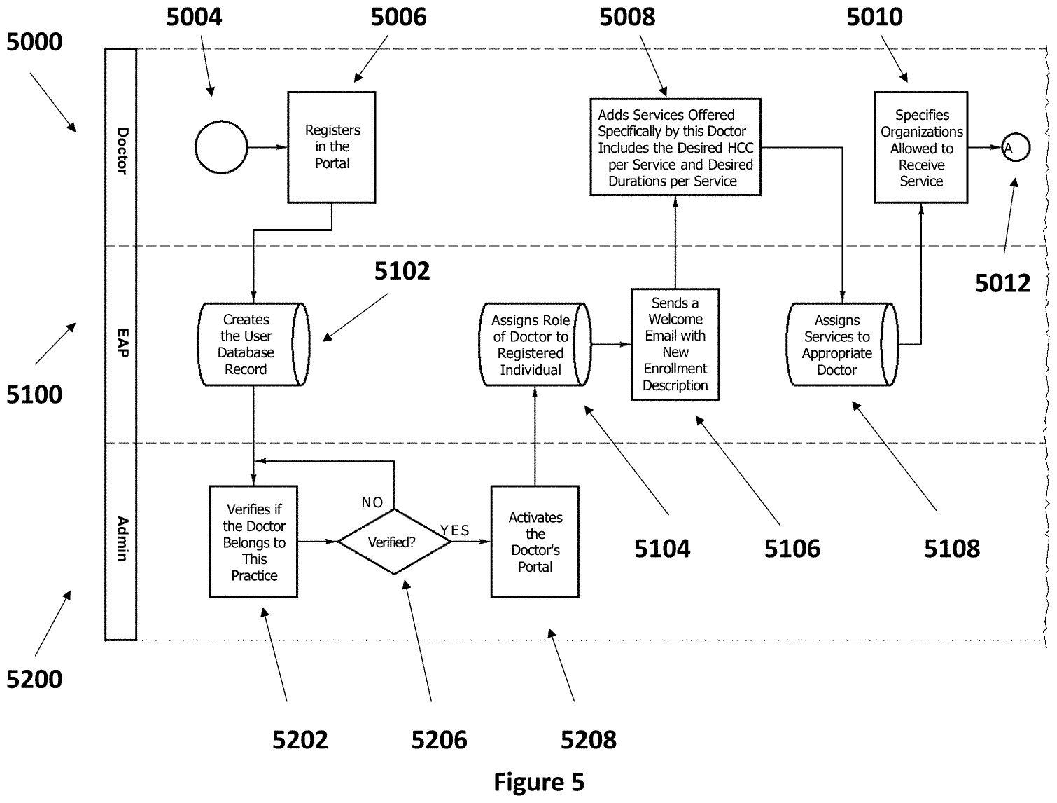

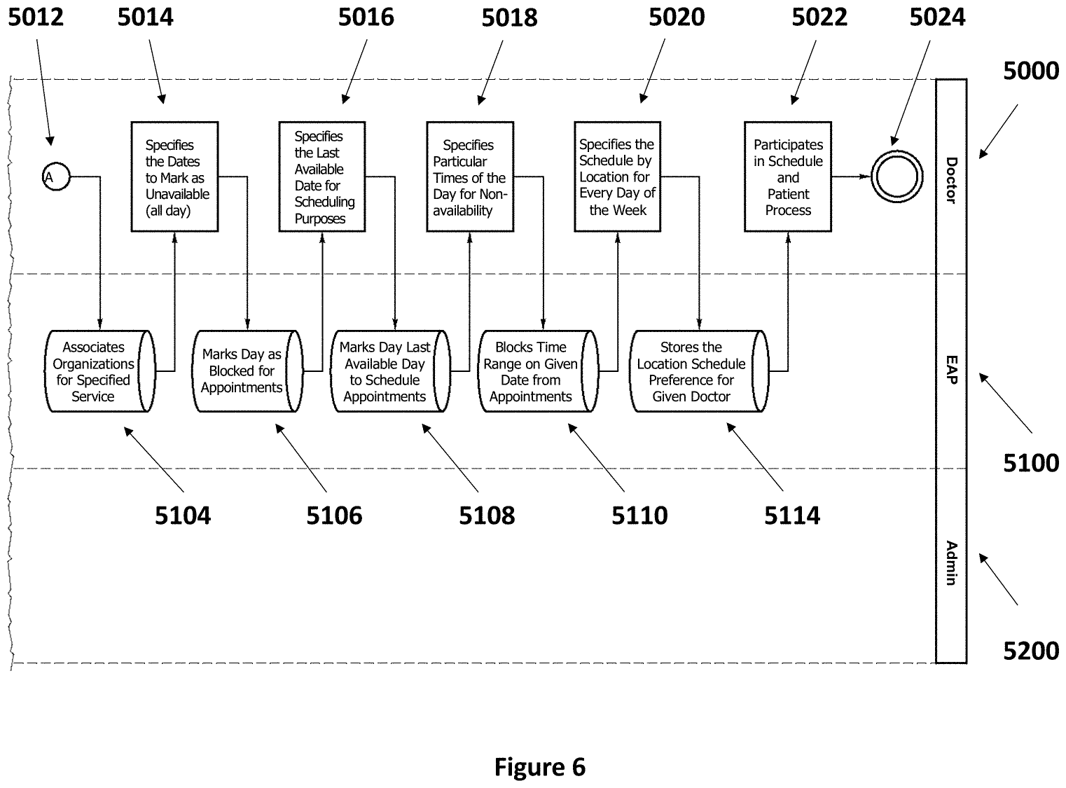

FIGS. 5-6 illustrate an aspect of this embodiment depicted as three different layers: the Expert/Doctor level 5000; the EAP level 5100; and the Admin layer 5200.

The Expert/Doctor level 5000 focuses on the ability of an expert to define the services to offer, the rate at which those services are offered, where to offer the services and on what days of the week. Furthermore, as FIGS. 5-6 help illustrate, the expert/doctor is able to block particular full or partial days.

The process of this embodiment starts at point 5004 that leads to a user registering the Portal 5006, that leads to a database creating the user in a record 5102. The admin layer 5200 then verifies if a user selected doctor belongs to the practice within the system 5202. If verified 5206, the system then activates the doctor's portal in module 5208, and thus assigns a role to the doctor to the registered user 5104 in EAP layer 5100. The process then sends a welcome email with the email description 5106 to the user. The process then adds any services offered by the doctor the desired HCC per service and desired durations of time per each service 5008. The process then assigns services to the appropriate doctor 5108 and then specifies organizations allowed to receive services 5010. FIG. 5 then indicates where the process leaves the figure at 5012 and then concludes in FIG. 6 at the same point 5012.

FIG. 6 then starts at point 5012 where FIG. 5 left off. The process starts in this figure by associating organizations for specified services 5104, then in turn specifies dates to mark as unavailable 5014. The process then marks the appropriate days as blocked for appointments 5106. The process then specifies the last available date for scheduling purposes 5015 and then marks the appropriate date as last available day to schedule appointments 5108. The process then specifies particular times of the day that are not available and then marks the appropriate times that are not available on those days 5114. Then the process ends 5024 when the doctor, or his/her staff, participates in scheduling the patient 5022.

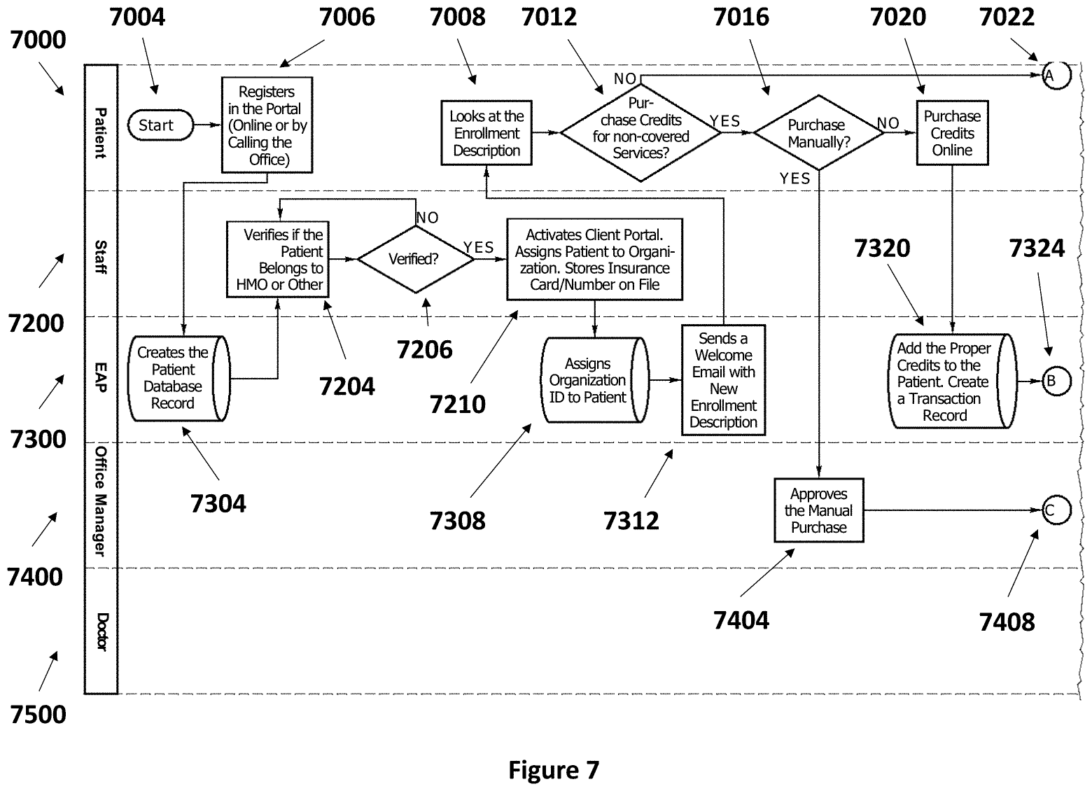

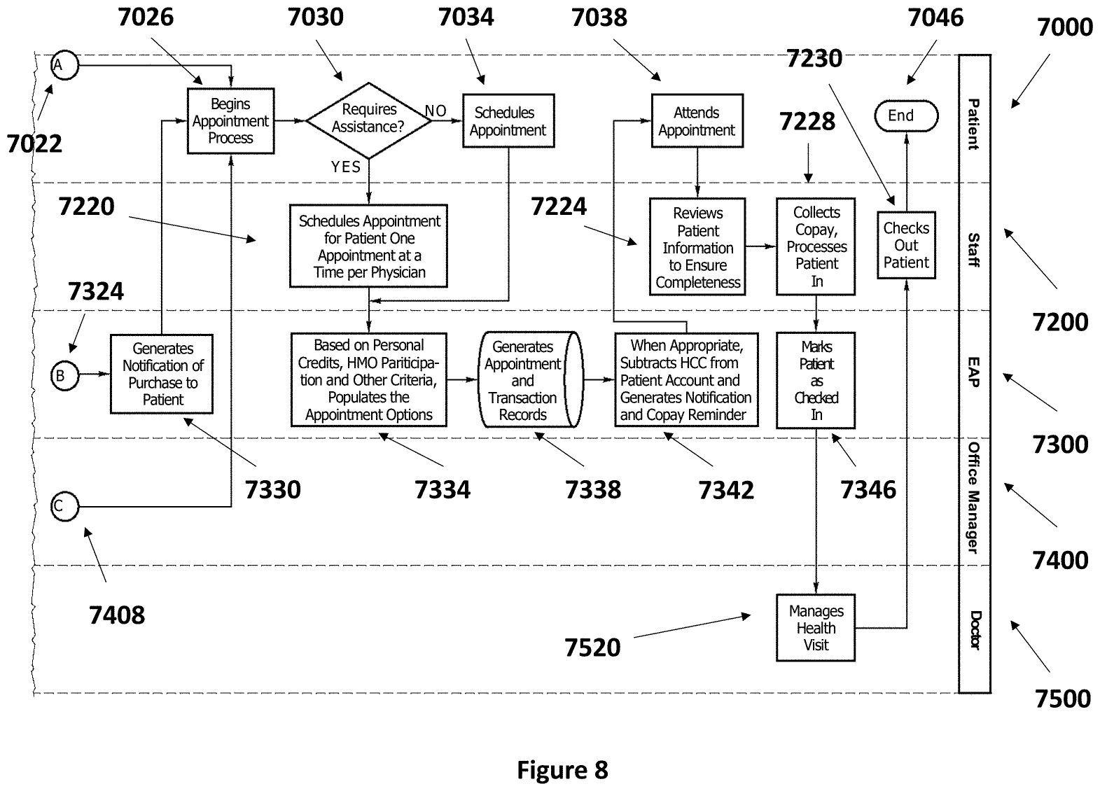

FIGS. 7-8 illustrate other aspects of the invention in 5 layers that include the original Doctor layer, but for ease of illustration, is now denoted as layer 7500 and the original EAP layer, as now denoted as layer 7300. Additional layers are added in this figure to represent patient layer 7000, a staff layer 7200, and an office manager layer 7400.

These FIGS. 7-8 focuses on the ability of a client to establish an account, purchase credits, determine doctor preference and schedule appointments online without day/time restriction.

FIG. 7 depicts that after the process starts 7004, a patient registers in the portal (online or by calling the office) 7006. The process then creates a record in the patient database 7304 and then tries to verify if the patient belongs to a HMO or other medical plan 7204. If verified 7206, the process then activates the portal to assign the patient to an organization and stores insurance information 7210. The process then assigns organization ID to the patient 7308. The process then sends a welcome email with the appropriate enrollment description to the patient 7312. The patient then looks at the enrollment description 7008 and decides whether to purchase credits on non-covered services 7012. If patient purchases credits manually 7016, then the office manager approves the manual purchase 7404 and then proceeds to end point 7408. If the patient purchases credits online 7020, the process then adds the proper credits to the patient by creating a transaction record 7320 and then the process leaves FIG. 7 at end point 7324.

In addition, if the patient decides not to purchase credits, the process ends in this figure at end point 7022.

Now referring to FIG. 8, if the process starts at either end point 7022 or end point 7408, the appointment process begins 7026. In addition, if the patient purchased credits online, the process first generates notification of the purchase for the patient 7330, but then also begins the appointment process 7026. After the appointment process begins 7026, the system determines if the patient needs assistance 7030, the process schedules one appointment at a time per each physician 7220. If no assistance is needed, an appointment is scheduled 7034. Either way, the process then proceeds to populate the appointment options based on personal credits, HMO participation and other criteria 7334. The appointment is then generated and a transaction record is created 7338. Then, when appropriate, the process subtracts the HCC from the patient account and generates a notification and copay reminder 7342.

Once the patient is ready for the appointment 7038, the process reviews the patient information to ensure it is complete 7224. The staff then collects the copay and processes the patient into the appointment 7228. The system then marks the patient present at the appointment 7346. The doctor then manages the health visit 7520 (appointment). The staff then checks out the patient of the appointment 7230 and ends 7046 the process.

While the process for obtaining a web visit is the same as requesting other visits, it is important to note, in this embodiment, that an additional interface exists for proper management of the web visit. However, due to the nature of the visit, additional ports may be added.

In this embodiment, when web visits are requested via the EAP Portal, and a link becomes available a few minutes before the appointment. The extra time provides the client with enough time to set up and be ready for the visit.

Additionally, this embodiment is designed with the following constructs described in the next 4 paragraphs.

Only 2 entities are allowed on that visit: 1 expert and 1 client. This design construct accounts for clients in the healthcare industry.

Only the doctor/expert is able to initiate the communication. This means that the while the client is able to enter the virtual room at any time, only the expert is able to establish the connection between him/her and the client.

Both expert and client are able to exit the room at any time for any reason.

The client will be able to reenter the room as long as the expert is available for that virtual room.

Although this invention has been described with reference to an illustrative embodiment, this description is not intended to limit the scope of the invention. Various modifications and combinations of the illustrative embodiments as well as other embodiments of the invention will be apparent to persons skilled in the art upon reference to the description. It is therefore intended that the appended claims accomplish any such modifications or embodiments

We claim:

1. A method for a user to register and schedule an appointment with a medical professional, the method comprising:
registering a patient user;
collecting and managing patient information;
collecting and managing doctor information, wherein the doctor information includes services offered, locations for offered services and unavailable dates;
storing medical insurance plans;
storing types of visits and locations of visits by type; and
storing and managing credits available for purchase by the patient user.

2. The method of claim 1, wherein the collecting and managing doctor information is performed within a database that also includes storing types of visits.

3. A portal for registering users and scheduling appointment for the users with medical professionals, the portal comprising:
an interface for registering a patient user;
a patient database for collecting and managing patient information;
a doctor database for collecting and managing doctor information, wherein the doctor information includes services offered, locations for offered services and unavailable dates;
a insurance database for storing medical insurance plans;
a visit database for storing records that include types of visits and locations of visits by type; and
a credit database for storing and managing credits available for purchase by the patient user.

4. The portal of claim 3, wherein the doctor database and visit database are sub-databases within a single database.
